(19) United States
(12) Patent Application Publication
(10) Pub. No.: US 2024/0008787 A1
(43) Pub. Date: Jan. 11, 2024

(54) FOOT STRENGTH MEASURING ASSEMBLY

(71) Applicant: Mike Salomon, Huntington Beach, CA (US)

(72) Inventor: Mike Salomon, Huntington Beach, CA (US)

(21) Appl. No.: 17/860,905

(22) Filed: Jul. 8, 2022

(51) Int. Cl. A61B 5/22; A61B 5/00

(52) U.S. Cl. CPC A61B 5/224; A61B 5/6829; A61B 5/6831; A61B 5/742; A61B 2562/0247; A61B 2562/043; A61B 2560/0214

(57) ABSTRACT

A foot strength measuring assembly includes a base is positionable on a horizontal support surface. A panel is pivotally disposed on the base thereby facilitating a user to position each of their feet on the panel. A tensioning unit is integrated into the base to adjust an amount of force necessary to pivot the panel on the base thereby requiring the user to apply a pre-determined amount of pressure to the panel to tilt the panel on the base. A plurality of pressure sensors is each of the pressure sensors is integrated into the panel to sense the magnitude of force applied to the panel when the user attempts to pivot the panel with their feet. A diagnostic unit is integrated into the panel to communicate the magnitude of pressure sensed by the plurality of pressure sensors to facilitate a caregiver to diagnose the function of the user's feet.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to foot strength device and more particularly pertains to a new foot strength device for diagnosing foot strength in a user. The device includes a base and a panel pivotally disposed on the base and a user positions their feet on the panel to tilt the panel. The device includes pressure sensors integrated into the panel and a diagnostic unit integrated into the base for analyzing data collected from the pressure sensors. The device includes a tensioning unit integrated into the base for adjusting the degree of force necessary to pivot the panel on the base.

### (2) Description of Related Art Including Information Disclosed Under 37 CFR 1.97 and 1.98

The prior art relates to foot strength devices including a compression device which is in communication with an external diagnostic device for testing a user's strength. The prior art discloses a strength measuring device that includes a lever arm that is manipulated by a user. The prior art discloses a foot strength device that includes a panel and a member that is movably spaced from the panel for measuring force required to move the member. The prior art discloses a strength meter that includes load cells for measuring force applied to the load cells and a belt travelling between a plurality of gears. The prior art discloses a foot strength meter that includes a base panel, an armature attached to the base panel and a foot saddle coupled to the armature for measuring the force required to tilt the foot saddle. The prior art discloses a toe strength meter that includes a panel, a toe strap that is positioned over a user's toe and a signal analyzer to analyze the flexion strength of a user's toe.

### BRIEF SUMMARY OF THE INVENTION

An embodiment of the disclosure meets the needs presented above by generally comprising a base is positionable on a horizontal support surface. A panel is pivotally disposed on the base thereby facilitating a user to position each of their feet on the panel. A tensioning unit is integrated into the base to adjust an amount of force necessary to pivot the panel on the base thereby requiring the user to apply a pre-determined amount of pressure to the panel to tilt the panel on the base. A plurality of pressure sensors is each of the pressure sensors is integrated into the panel to sense the magnitude of force applied to the panel when the user attempts to pivot the panel with their feet. A diagnostic unit is integrated into the panel to communicate the magnitude of pressure sensed by the plurality of pressure sensors to facilitate a caregiver to diagnose the function of the user's feet.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a top perspective view of a foot strength measuring assembly according to an embodiment of the disclosure.

FIG. 2 is a top perspective in-use view of an embodiment of the disclosure.

FIG. 3 is a left side in-use view of an embodiment of the disclosure.

FIG. 4 is a schematic view of an embodiment of the disclosure.

FIG. 5 is a top phantom view of a base of an embodiment of the disclosure.

### DETAILED DESCRIPTION OF THE INVENTION

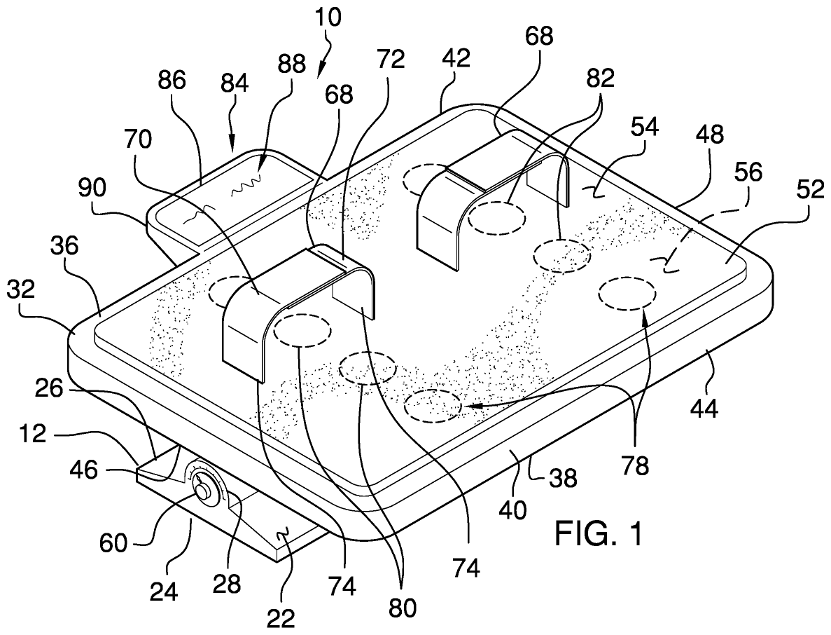

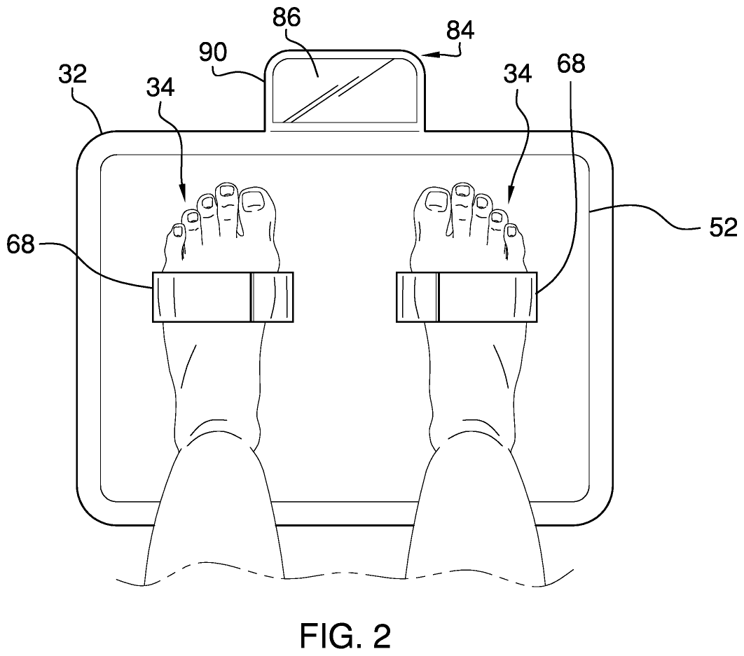

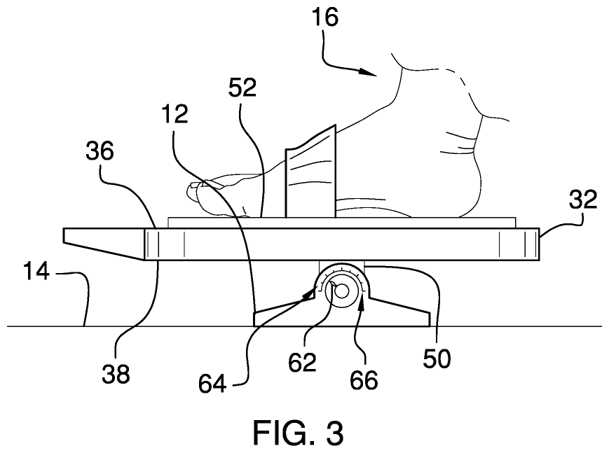

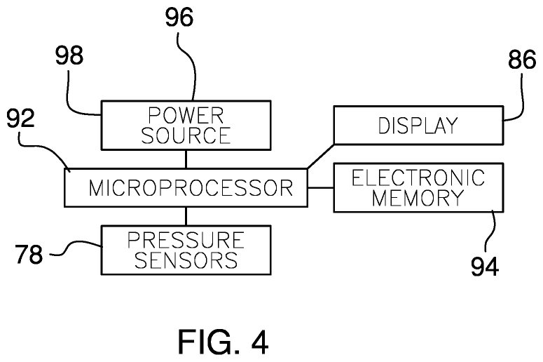

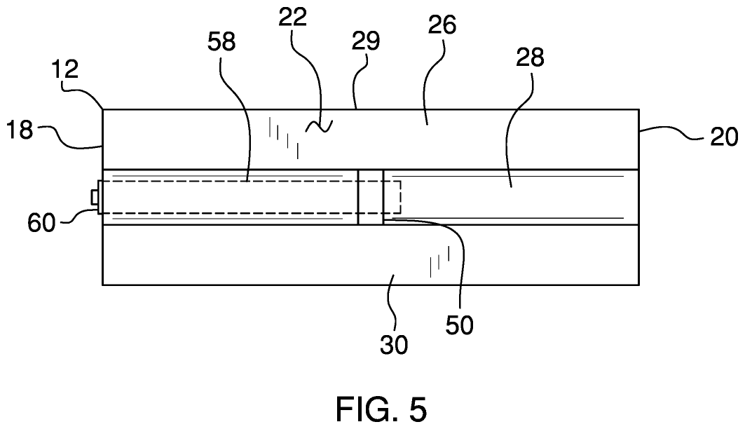

With reference now to the drawings, and in particular to FIGS. 1 through 5 thereof, a new foot strength device embodying the principles and concepts of an embodiment of the disclosure and generally designated by the reference numeral 10 will be described.

As best illustrated in FIGS. 1 through 5, the foot strength measuring assembly 10 generally comprises a base 12 that is positionable on a horizontal support surface 14. The horizontal support surface 14 may be a floor or other support surface 14 upon which a user 16 might be standing. The base 12 has a first end 18, a second end 20 and an outer surface 22 extending between the first end 18 and the second end 20, and the outer surface 22 has a lower side 24 and an upper side 26. Additionally, the base 12 is elongated between the first end 18 and the second end 20, and the lower side 24 is planar thereby facilitating the lower side 24 to rest on the horizontal support surface 14. The upper side 26 has a lobe 28 extending upwardly from the upper side 26 and the lobe 28 is centrally positioned on the upper side 26. Furthermore, the lobe 28 extends between the first end 18 and the second end 20. The upper side 26 slopes downwardly between the lobe 28 and a respective front edge 29 and rear edge 30 of the base 12.

A panel 32 is provided and the panel 32 is pivotally disposed on the base 12 thereby facilitating a user 16 to position each of their feet 34 on the panel 32. The panel 32 has a top side 36, a bottom side 38 and a perimeter edge 40 extending between the top side 36 and the bottom side 38, and the perimeter edge 40 has a front side 42, a back side 44, a first lateral side 46 and a second lateral side 48. The panel 32 has a pivot 50 extending downwardly from the bottom side 38, the pivot 50 pivotally engages the lobe 28 on the upper side 26 of the base 12 for pivotally attaching the panel 32 to the base 12 and the pivot 50 is centrally positioned on the bottom side 38.

A pad 52 is provided that has a lower surface 54 and an upper surface 56, and the lower surface 54 is bonded to the top side 36 of the panel 32 thereby facilitating the user 16 to place their feet 34 on the upper surface 56 of the pad 52. The pad 52 covers a substantial area of the top side 36 of the panel 32 and the pad 52 is comprised of a resiliently compressible material, including but not being limited to rubber, thereby enhancing comfort for the user 16.

A tensioning unit 58 is integrated into the base 12 and the tensioning unit 58 is in mechanical communication with the panel 32. The tensioning unit 58 is adjustable between a minimum tension and a maximum tension thereby adjusting an amount of force necessary to pivot 50 the panel 32 on the base 12. In this way the user 16 is required to apply a pre-determined amount of pressure to the panel 32 to tilt the panel 32 on the base 12. Additionally, the tensioning unit 58 engages the pivot 50 on the bottom side 38 of the base 12. The tensioning unit 58 may comprise an electro-mechanical tensioner that poses rotational resistance against the pivot 50 on the base 12, the tensioning unit 58 may comprise an adjustable spring mechanism that poses adjustable resistance against the pivot 50 or the tensioning unit 58 may comprise any mechanism that can resist rotation of the pivot 50 in the base 12.

The tensioning unit 58 including a knob 60 that is rotatably disposed on the first end 18 of the base 12. The knob 60 is rotatable between a minimum position and a maximum position for adjusting the tensioning unit 58 between the minimum tension and the maximum tension. An indicating arrow 62 is printed on the knob 60 and the base 12 has indicator indicia 64 printed on the first end 18 of the base 12. The indicator indicia 64 comprise a graduated scale 66 extending around a substantial circumference of the knob 60 thereby facilitating the indicating arrow 62 on the knob 60 to be aligned with a desired location along the graduated scale 66 for adjusting the tension of the tensioning unit 58.

A pair of foot straps 68 is each disposed on the panel 32 thereby facilitating each of the straps to be extended over a respective one of the user's feet 34 for securing the user's feet 34 to the panel 32. Each of the foot straps 68 includes a first portion 70 and a second portion 72, and each of the first portion 70 and the second portion 72 of each of the foot straps 68 has a coupled end 74 that is coupled to the upper surface 56 of the pad 52. Each of the first portion 70 and the second portion 72 of a respective foot strap 68 is aligned with each other. Additionally, each of the first portion 70 and the second portion 72 of the respective foot strap 68 is matable to each other to secure the respective user 16's foot to the pad 52. Each of the foot straps 68 is spaced from a respective first lateral side 46 and the second lateral side 48 of the perimeter edge 40 of the panel 32, and each of the foot straps 68 is positioned closer to the front side 42 than the back side 44 of the perimeter edge 40 of the panel 32. Each of the first portion 70 and the second portion 72 of each of the foot straps 68 may include a mating member, such as a hook and loop fastener or the like, thereby facilitating the first portion 70 and the second portion 72 of each of the foot straps 68 to be releasably mated to each other.

A plurality of pressure sensors 78 is provided and each of the pressure sensors 78 is integrated into the panel 32. Each of the pressure sensors 78 is strategically positioned such that each of the pressure sensors 78 is positioned beneath a respective one of the user's feet 34 when the user's feet 34 are positioned on the panel 32. In this way each of the pressure sensors 78 can sense the magnitude of force applied to the panel 32 when the user 16 attempts to pivot 50 the panel 32 with their feet 34. Each of the pressure sensors 78 is integrated into the top side 36 of the panel 32 such that each of the pressure sensors 78 is positioned beneath the pad 52. Furthermore, each of the pressure sensors 78 may comprise an electronic pressure sensor with a sensitivity ranging between approximately 0.5 kg and 11.0 kg.

The plurality of pressure sensors 78 including a set of first pressure sensors 80 and a set of second pressure sensors 82. Each of the first pressure sensors 80 and the second pressure sensors 82 is spaced apart from each other and is distributed between the front side 42 and the back side 44 of the perimeter edge 40 of the panel 32. Additionally, each of the first pressure sensors 80 and the second pressure sensors 82 is spaced from a respective one of the first lateral side 46 and the second lateral side 48 of the perimeter edge 40 of the panel 32. Each of the first pressure sensors 80 and the second pressure sensors 82 extends between the first portion 70 and the second portion 72 of a respective one of the foot straps 68.

A diagnostic unit 84 is integrated into the panel 32 and the diagnostic unit 84 is in communication with each of the pressure sensors 78. The diagnostic unit 84 includes a display 86 that is disposed on the panel 32 thereby facilitating the display 86 to be visible to the user 16. The display 86 displays indicia 88 comprising numbers to communicate the magnitude of pressure sensed by the plurality of pressure sensors 78. In this way the diagnostic unit 84 facilitates a caregiver to diagnose the function of the user's feet 34. The caregiver may be a doctor, a chiropractor or other individual that is diagnosing a potential foot injury suffered by the user 16.

The display 86 extends away from the front side 42 of the perimeter edge 40 of the panel 32 such that the display 86 lies on a plane is coplanar with the top side 36 of the panel 32. Additionally, the display 86 is centrally positioned between the first lateral side 46 and the second lateral side 48 of the perimeter edge 40 of the panel 32. The display 86 may comprise a liquid crystal display or the like and the display 86 may include a housing 90 that extends away from the front side 42 of the perimeter edge 40 of the panel 32 into which the display 86 is integrated.

The diagnostic unit 84 comprises a control circuit 92 that is integrated into the panel 32 and each of the pressure sensors 78 is electrically coupled to the control circuit 92. Additionally, the display 86 is electrically coupled to the control circuit 92 and the control circuit 92 is electrically coupled to the tensioning unit 58. An electronic memory 94 is integrated into the panel 32 and the electronic memory 94 is electrically coupled to the control circuit 92. The electronic memory 94 stores operational software comprising a diagnostic program for analyzing data collected from the pressure sensors 78. The display 86 displays the indicia 88 to communicate a diagnosis generated by the operational software. The diagnostic unit 84 includes a power supply 96 that is integrated into the panel 32, the power supply 96 is electrically coupled to the control circuit 92 and the power supply 96 comprising at least one battery 98.

In use, the user 16 positions each of their feet 34 on the pad 52 and each of the foot straps 68 is secured over a respective one of the user's feet 34 to secure the user's feet 34 to the pad 52. In this way each of the user's feet 34 is positioned over the first pressure sensors 80 and the second pressure sensors 82. The knob 60 is adjusted to set the tensioning unit 58 to the desired tension and the user 16 rocks their feet 34 either forwardly or rearwardly on the panel 32 to either tilt the front side 42 or the back side 44 of the perimeter edge 40 of the panel 32 downwardly. Additionally, each of the pressure sensors 78 records the magnitude of force that is imparted into the panel 32. The diagnostic unit 84 analyzes the data collected by the pressure sensors 78 and the display 86 displays the indicia 88 to facilitate the caregiver to diagnose the user 16. In this way the caregiver can diagnose an injury that the user 16 has suffered, or the caregiver can monitor the progression of healing the user 16 has made with respect to a previous injury or corrective surgery performed on the user's feet 34.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A foot strength measuring assembly for measuring the strength of a user's foot for diagnostic purposes, said assembly comprising:
a base being positionable on a horizontal support surface;
a panel being pivotally disposed on said base thereby facilitating a user to position each of their feet on said panel;
a tensioning unit being integrated into said base, said tensioning unit being in mechanical communication with said panel, said tensioning unit being adjustable between a minimum tension and a maximum tension thereby adjusting an amount of force necessary to pivot said panel on said base thereby requiring the user to apply a pre-determined amount of pressure to said panel to tilt said panel on said base;
a pair of foot straps, each of said foot straps being disposed on said panel thereby facilitating each of said straps to be extended over a respective one of the user's feet for securing the user's feet to said panel;
a plurality of pressure sensors, each of said pressure sensors being integrated into said panel, each of said pressure sensors being strategically positioned such that each of said pressure sensors is positioned beneath a respective one of the user's feet when the user's feet are positioned on said panel thereby facilitating each of said pressure sensors to sense the magnitude of force applied to said panel when the user attempts to pivot said panel with their feet; and
a diagnostic unit being integrated into said panel, said diagnostic unit being in communication with each of said pressure sensors, said diagnostic unit including a display being disposed on said panel thereby facilitating said display to be visible to the user, said display displaying indicia comprising numbers to communicate the magnitude of pressure sensed by said plurality of pressure sensors wherein said diagnostic unit is configured to facilitate a caregiver to diagnose the function of the user's feet.

2. The assembly according to claim 1, wherein said base has a first end, a second end and an outer surface extending between said first end and said second end, said outer surface having a lower side and an upper side, said base being elongated between said first end and said second end, said lower side being planar thereby facilitating said lower side to rest on the horizontal support surface, said upper side having a lobe extending upwardly from said upper side, said lobe being centrally positioned on said upper side, said lobe extending between said first end and said second end, said upper side sloping downwardly between said lobe and a respective front edge and rear edge of said base.

3. The assembly according to claim 2, wherein said panel has a top side, a bottom side and a perimeter edge extending between said top side and said bottom side, said perimeter edge having a front side, a back side, a first lateral side and a second lateral side, said panel having a pivot extending downwardly from said bottom side, said pivot pivotally engaging said lobe on said upper side of said base for pivotally attaching said panel to said base, said pivot being centrally positioned on said bottom side.

4. The assembly according to claim 2, further comprising a pad having a lower surface and an upper surface, said lower surface being bonded to said top side of said panel thereby facilitating the user to place their feet on said upper surface of said pad, said pad covering a substantial area of said top side of said panel.

5. The assembly according to claim 3, wherein:
said tensioning unit engages said pivot on said bottom side of said base;
said tensioning unit includes a knob being rotatably disposed on said first end of said base, said knob being rotatable between a minimum position and a maximum position for adjusting said tensioning unit between said minimum tension and said maximum tension;
said knob has an indicating arrow being printed thereon;
said base has indicator indicia being printed on said first end of said base, said indicator indicia comprising a graduated scale extending around a substantial circumference of said knob thereby facilitating said indicating arrow on said knob to be aligned with a desired location along said graduated scale for adjusting the tension of said tensioning unit.

6. The assembly according to claim 4, wherein each of said foot straps includes a first portion and a second portion, each of said first portion and said second portion of each of said foot straps having a coupled end being coupled to said upper surface of said pad, each of said first portion and said second portion of a respective foot strap being aligned with each other, each of said first portion and said second portion of said respective foot strap being matable to each other to secure the respective user's foot to said pad, each of said foot straps being spaced from a respective first lateral side and said second lateral side of said perimeter edge of said panel, each of said foot straps being positioned closer to said front side than said back side of said perimeter edge of said panel.

7. The assembly according to claim 6, wherein:
each of said pressure sensors is integrated into said top side of said panel such that each of said pressure sensors is positioned beneath said pad;
said plurality of pressure sensors includes a set of first pressure sensors and a set of second pressure sensor;
each of said first pressure sensors and said second pressure sensors is spaced apart from each other and is distributed between said front side and said back side of said perimeter edge of said panel, each of said first pressure sensors and said second pressure sensors being spaced from a respective one of said first lateral side and said second lateral side of said perimeter edge of said panel; and
each of said first pressure sensors and said second pressure sensors extends between said first portion and said second portion of a respective one of said foot straps.

8. The assembly according to claim 3, wherein said display extends away from said front side of said perimeter edge of said panel such that said display lies on a plane being coplanar with said top side of said panel, said display being centrally positioned between said first lateral side and said second lateral side of said perimeter edge of said panels

9. The assembly according to claim 1, wherein said diagnostic unit comprises:
a control circuit being integrated into said panel, each of said pressure sensors being electrically coupled to said control circuit, said display being electrically coupled to said control circuit, said control circuit being electrically coupled to said tensioning unit;
an electronic memory being integrated into said panel, said electronic memory being electrically coupled to said control circuit, said electronic memory storing operational software comprising a diagnostic program for analyzing data collected from said pressure sensors, said display displaying indicia to communicate a diagnosis generated by said operational software; and
a power supply being integrated into said panel, said power supply being electrically coupled to said control circuit, said power supply comprising at least one battery.

10. A foot strength measuring assembly for measuring the strength of a user's foot for diagnostic purposes, said assembly comprising:
a base being positionable on a horizontal support surface, said base having a first end, a second end and an outer surface extending between said first end and said second end, said outer surface having a lower side and an upper side, said base being elongated between said first end and said second end, said lower side being planar thereby facilitating said lower side to rest on the horizontal support surface, said upper side having a lobe extending upwardly from said upper side, said lobe being centrally positioned on said upper side, said lobe extending between said first end and said second end, said upper side sloping downwardly between said lobe and a respective front edge and rear edge of said base;
a panel being pivotally disposed on said base thereby facilitating a user to position each of their feet on said panel, said panel having a top side, a bottom side and a perimeter edge extending between said top side and said bottom side, said perimeter edge having a front side, a back side, a first lateral side and a second lateral side, said panel having a pivot extending downwardly from said bottom side, said pivot pivotally engaging said lobe on said upper side of said base for pivotally attaching said panel to said base, said pivot being centrally positioned on said bottom side;
a pad having a lower surface and an upper surface, said lower surface being bonded to said top side of said panel thereby facilitating the user to place their feet on said upper surface of said pad, said pad covering a substantial area of said top side of said panel;
a tensioning unit being integrated into said base, said tensioning unit being in mechanical communication with said panel, said tensioning unit being adjustable between a minimum tension and a maximum tension thereby adjusting an amount of force necessary to pivot said panel on said base thereby requiring the user to apply a pre-determined amount of pressure to said panel to tilt said panel on said base, said tensioning unit engaging said pivot on said bottom side of said base, said tensioning unit including a knob being rotatably disposed on said first end of said base, said knob being rotatable between a minimum position and a maximum position for adjusting said tensioning unit between said minimum tension and said maximum tension, said knob having an indicating arrow being printed thereon, said base having indicator indicia being printed on said first end of said base, said indicator indicia comprising a graduated scale extending around a substantial circumference of said knob thereby facilitating said indicating arrow on said knob to be aligned with a desired location along said graduated scale for adjusting the tension of said tensioning unit;
a pair of foot straps, each of said foot straps being disposed on said panel thereby facilitating each of said straps to be extended over a respective one of the user's feet for securing the user's feet to said panel, each of said foot straps including a first portion and a second portion, each of said first portion and said second portion of each of said foot straps having a coupled end being coupled to said upper surface of said pad, each of said first portion and said second portion of a respective foot strap being aligned with each other, each of said first portion and said second portion of said respective foot strap being matable to each other to secure the respective user's foot to said pad, each of said foot straps being spaced from a respective first lateral side and said second lateral side of said perimeter edge of said panel, each of said foot straps being positioned closer to said front side than said back side of said perimeter edge of said panel;
a plurality of pressure sensors, each of said pressure sensors being integrated into said panel, each of said pressure sensors being strategically positioned such that each of said pressure sensors is positioned beneath a respective one of the user's feet when the user's feet are positioned on said panel thereby facilitating each of said pressure sensors to sense the magnitude of force applied to said panel when the user attempts to pivot said panel with their feet, each of said pressure sensors being integrated into said top side of said panel such that each of said pressure sensors is positioned beneath said pad, said plurality of pressure sensors including a set of first pressure sensors and a set of second pressure sensors, each of said first pressure sensors and said second pressure sensors being spaced apart from each other and being distributed between said front side and said back side of said perimeter edge of said panel, each of said first pressure sensors and said second pressure sensors being spaced from a respective one of said first lateral side and said second lateral side of said perimeter edge of said panel, each of said first pressure sensors and said second pressure sensors extending between said first portion and said second portion of a respective one of said foot straps; and
a diagnostic unit being integrated into said panel, said diagnostic unit being in communication with each of said pressure sensors, said diagnostic unit including a display being disposed on said panel thereby facilitating said display to be visible to the user, said display displaying indicia comprising numbers to communicate the magnitude of pressure sensed by said plurality of pressure sensors wherein said diagnostic unit is configured to facilitate a caregiver to diagnose the function of the user's feet, said display extending away from said front side of said perimeter edge of said panel such that said display lies on a plane being coplanar with said top side of said panel, said display being centrally positioned between said first lateral side and said second lateral side of said perimeter edge of said panel, said diagnostic unit comprising:
a control circuit being integrated into said panel, each of said pressure sensors being electrically coupled to said control circuit, said display being electrically coupled to said control circuit, said control circuit being electrically coupled to said tensioning unit;
an electronic memory being integrated into said panel, said electronic memory being electrically coupled to said control circuit, said electronic memory storing operational software comprising a diagnostic program for analyzing data collected from said pressure sensors, said display displaying indicia to communicate a diagnosis generated by said operational software; and
a power supply being integrated into said panel, said power supply being electrically coupled to said control circuit, said power supply comprising at least one battery.
